(19) United States
(12) Patent Application Publication
(10) Pub. No.: US 2022/0031240 A1
(43) Pub. Date: Feb. 3, 2022

(54) INTEGRATED BIO-PATCH AND BIO-BRACELET SYSTEMS

(71) Applicant: LIFE PATCH INTERNATIONAL, Irvine, CA (US)

(72) Inventors: Joseph P. FEEHAN, Orlando, FL (US); Donald BOLLELLA, Irvine, CA (US)

(21) Appl. No.: 17/386,136

(22) Filed: Jul. 27, 2021

Related U.S. Application Data
(60) Provisional application No. 63/057,860, filed on Jul. 28, 2020.

(51) Int. Cl. A61B 5/00

(52) U.S. Cl. CPC A61B 5/681; A61B 5/6831; A61B 5/6829; A61B 5/6823; A61B 5/6828; A61B 5/6824

(57) ABSTRACT

An integrated patch and bracelet system wearable on a user to perform a diagnostic function. The system includes a bracelet portion having a central housing member and a pair of extending straps. A socket is provided in a back side of the central housing member configured to receive a patch portion having a peripheral edge that conforms in shape to the socket. Reusable components are provided in the bracelet portion and consumable components are provided in the patch portion. In use, the patch portion is seated in register with the socket in the central housing member thereby allowing interoperability between at least one component in the patch portion and the components in the central housing member of the bracelet portion.

### CROSS-REFERENCE TO RELATED APPLICATION(S)

This application claims priority to U.S. Provisional Application No. 63/057,860 filed Jul. 28, 2020, entitled INTEGRATED BIO-PATCH AND BIO-BRACELET SYSTEMS, the disclosure of which is hereby expressly incorporated by reference herein in its respective entirety.

### BACKGROUND

### Field

The present disclosure relates in general to health and wellness and, in particular, to personal diagnostic devices. More specifically, but without restriction to the particular embodiments hereinafter described in accordance with the best mode of practice, the present disclosure relates a wearable two-piece integrated bio-patch and bio-bracelet system.

### Description of the Related Art

Health and wellness of individuals has been a concern since the dawn of time and with the advent of micro and nano-technologies beginning in the twentieth century there is presented an opportunity to merge advances from several different arts into a wearable personal diagnostic device including nano-electronics, bio-chemistries, and micro-mechanics. Such devices may include one-time or short-term use components such as those used for clinical diagnostics and other long-term components such as those used for electronic or optical signal processing. Thus there is a need to provide a two-piece wearable diagnostic system for personal use where the consumable components are provided in a first assembly and the reusable components are provided in a second assembly such that the two assemblies working together provide an integrated functional device that performs its intended results.

### SUMMARY

In accordance with some implementations, the present disclosure relates to an integrated patch and bracelet system wearable on a user. The system includes a bracelet portion including a central housing member and a pair of straps extending therefrom with each strap having an end, with the central housing member having a socket formed in a back side thereof and configured to contain a plurality of electronic components. The system further includes a patch portion configured to contain at least one diagnostic component to perform a diagnostic function and having a peripheral edge that conforms in shape to the socket formed in the central housing member so that when the patch portion is applied to a skin surface of a user and the bracelet portion is positioned over the patch portion with the ends of the straps secured together around a body part of the user, the patch portion is seated in register with the socket in the central housing member thereby allowing interoperability between the at least one diagnostic component in the patch portion and at least one electronic component in the central housing member.

In some embodiments, the system can further include a dimplet formed in an upper surface of the socket of the central housing member. In some embodiments, the system can further include a dome section formed on a top surface of the patch portion, with the dome section containing therein at least a portion of the at least one diagnostic component in the patch portion. In some embodiments, when the bracelet portion is positioned over the patch portion with the ends of the straps secured together around the body part of the user, the dome section of the patch portion can be seated in register with the dimplet formed in the socket in the central housing member thereby allowing interoperability between the portion of the at least one diagnostic component in the dome section and the at least one electronic component in the central housing member.

In some embodiments, the body part of the user can be associated with a body location such as, for example, a middle upper arm location, a high upper arm location, a lower upper arm location, an upper forearm location, a middle forearm location, a wrist location, a chest location, a mid-trunk location, an abdominal location, an upper thigh location, a mid-thigh location, a lower thigh location, an upper calf location, a mid-calf location, a lower calf location, or an ankle location.

In some implementations, the present disclosure relates to a wearable bracelet that includes a central housing member having a socket formed in a back side thereof and configured to contain a plurality of electronic components. The central housing member is further configured to receive a patch having a diagnostic component for providing a diagnostic function. The socket is dimensioned such that a peripheral edge of the patch substantially conforms in shape to the socket so that when the patch is applied to a skin surface of a user and the bracelet is positioned over the patch, the patch is seated in register with the socket in the central housing member thereby allowing interoperability between the diagnostic component in the patch and at least one electronic component in the central housing member. The bracelet further includes one or more straps extending from the central housing member and configured to allow the bracelet to be secured to a body part associated with the skin surface of the user.

In some embodiments, the central housing member can further include a dimplet formed in an upper surface of the socket. In some embodiments, the dimplet of the socket can be dimensioned to index with a dome section formed on a top surface of the patch.

In some implementations, the present disclosure relates to a patch that includes a diagnostic component for providing a diagnostic function. The patch has a shape that substantially conforms to a socket of a central housing member when the patch is seated in register with the socket, with the central housing member having a plurality of electronic components and being a part of a bracelet, so that when the patch is applied to a skin surface of a user and the bracelet is positioned over the patch, the patch being seated in register with the socket in the central housing member allows interoperability between the diagnostic component in the patch and at least one electronic component in the central housing member.

In some embodiments, the patch can further include a dome section formed on a top surface of the patch. In some embodiments, the dome section of the patch can be dimensioned to index with a dim plet formed in an upper surface of the socket of the central housing member.

For purposes of summarizing the disclosure, certain aspects, advantages and novel features of the inventions have been described herein. It is to be understood that not necessarily all such advantages may be achieved in accordance with any particular embodiment of the invention. Thus, the invention may be embodied or carried out in a manner that achieves or optimizes one advantage or group of advantages as taught herein without necessarily achieving other advantages as may be taught or suggested herein.

### BRIEF DESCRIPTION OF THE DRAWINGS

Various aspects of the present disclosure together with additional features contributing thereto and advantages accruing therefrom will be apparent from the following description of some embodiments of this disclosure which is shown in the accompanying drawing figures with like reference numerals indicating like components throughout, wherein:

FIG. 1 is a perspective view showing the back side of the bracelet portion of a two-piece wearable diagnostic system for personal use according to an embodiment of the present disclosure;

FIG. 2 is a plan view of the bracelet portion of FIG. 1;

FIG. 3 is a side view of the present two-piece system showing the bracelet portion of FIGS. 1 and 2 with a patch portion that conforms to a socket provided in the bracelet portion;

FIG. 4 is a side view of an alternate embodiment of the two-piece system of FIG. 3 showing a dimplet in the socket of the bracelet portion and a conforming dome section on the patch portion for secure seating and coupling between the two pieces;

FIG. 5 is a perspective view showing the back side of the bracelet portion of FIG. 4 and the conforming dome section on the corresponding patch portion in motion relative to each other to form the secure seating and coupling between the two pieces;

FIG. 6A is a pictorial representation of the patch portion of FIGS. 4 and 5 as placed for use on the forearm of a user of the present disclosure according to the teachings hereof;

FIG. 6B is a pictorial representation of the matching bracelet portion of FIGS. 4 and 5 shown in motion as being placed in alignment over the corresponding patch portion as initially placed for deployment in FIG. 6A on the forearm of the user of this embodiment of the present disclosure;

FIG. 6C is a pictorial representation of the two-piece system of FIGS. 6A and 6B shown in a final coupled condition ready for use as deployed on the forearm of the user in this particular body-placement example; and

FIG. 7 is a body schematic of a user of an embodiment the present disclosure showing several different body locations for deployment of the embodiment of the present disclosure according to the specific embodiment and application for each thereof.

### DETAILED DESCRIPTION OF SOME EMBODIMENTS

The headings provided herein, if any, are for convenience only and do not necessarily affect the scope or meaning of the claimed invention.

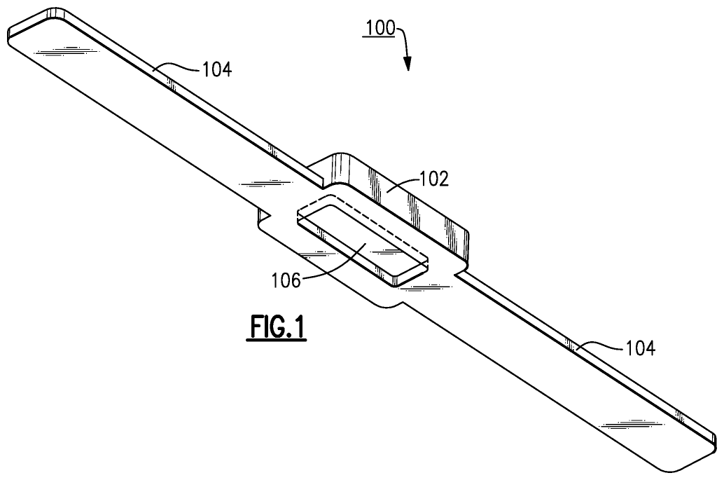

With reference now to FIG. 1, there is shown a perspective view of bracelet portion 100 of a two-piece wearable diagnostic system for personal use according to the present disclosure. The bracelet portion 100 includes a body, housing, or central member 102 and extending bands or straps 104. Further, the central member has formed therein a recess, receptacle, or socket 106.

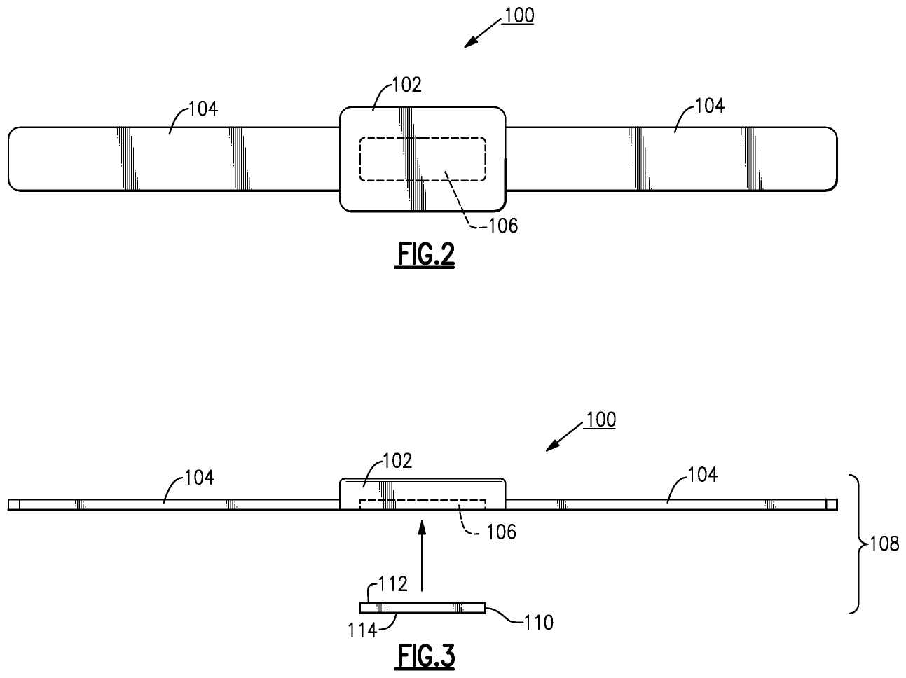

FIG. 2 is a plan view of the bracelet portion 100 of FIG. 1 showing the top side of the bracelet portion 100, the straps 104, and the socket 106 formed on the underside of the central member 102. The straps 104 may be of any desired length so as to fit comfortably around the body as suitable for the different embodiments hereof.

FIG. 3 is a side view of a two-piece system 108 according to the present disclosure showing the bracelet portion 100 of FIGS. 1 and 2 with a patch portion 110 that conforms to the socket 106 provided in the bracelet portion 100 as illustrated. The patch portion 110 has a top surface 112 and a bottom surface 114. The top surface 112 of the patch portion 110 is shaped along the periphery thereof to fit within the socket 106 of the bracelet portion 106 as illustrated. The bottom surface 114 of the patch portion 110 is provided with a suitable adhesive so that it may be applied to the skin of a user thereof. Thus in this manner, the two-piece system 108 allows a user of the system to first apply the patch portion 110 to a preferred location on the body such as the wrist, ankle, calf, thigh, forearm, upper arm, or around the torso, and then secure the bracelet portion 100 over the patch portion 110 with the straps 104 so that the patch portion 110 fits in register with the corresponding socket 106 in the bracelet portion 100. The patch portion 110 may be of the non-invasive type, the absorption type, or the fluidic type of patches disclosed in commonly assign U.S. Provisional Application Ser. No. 62/972,654 filed Feb. 11, 2020 titled Wearable Patches for Sports or U.S. Provisional Application Ser. No. 63/050,029 filed Jul. 9, 2020 titled Diagnostic Patches And Bracelets For Promoting Personal And Community Health Including Related Processes, Methods, And Systems which are both herein incorporated in their respective entities.

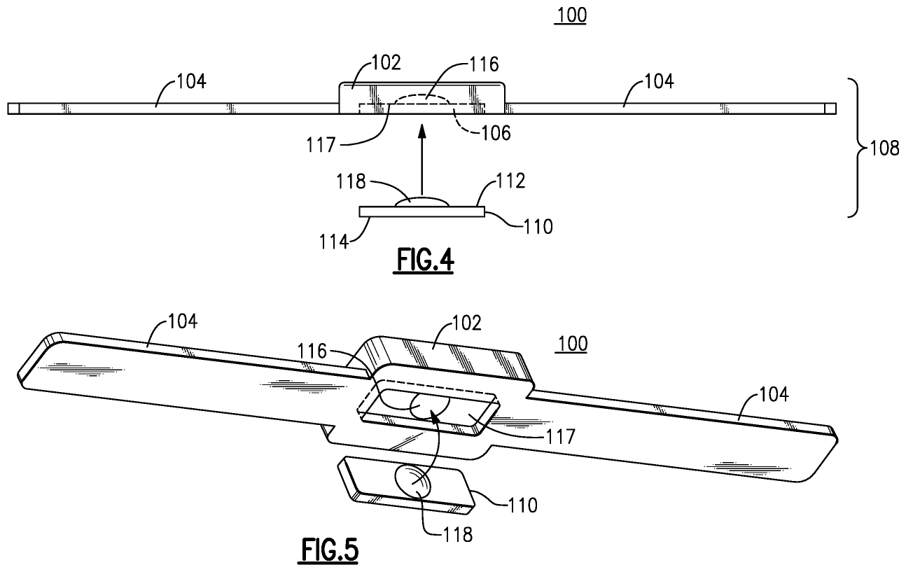

Referring next to FIG. 4, there is presented a side view of an alternate embodiment of the two-piece system 108 of FIG. 3 showing a dimplet 116 formed in an upper surface 117 in the socket 106 of the bracelet portion 100 and a conforming dome section 118 on the patch portion 110 for secure seating and coupling between the two pieces 100 and 110. In this manner, providing in-register seating between the patch portion 110 and the bracelet portion 100 is further facilitated by the mating of the dome section 118 with the dimplet 116 to thereby provide conformal contact between the two pieces 100 and 110.

FIG. 5 is a perspective view showing the back side of the bracelet portion 100 of FIG. 4 and the conforming dome section 118 on the corresponding patch portion 110 in motion relative to each other to form the secure seating and coupling between the two pieces 100 and 110. Thus there is provided a two-piece wearable diagnostic system for personal use where the consumable components may be implemented in the patch portion 110 and the reusable components such as electronics and such like are provided in the central member 102 of the bracelet portion 100 so that the two assemblies working together provide an integrated functional device that performs its intended results.

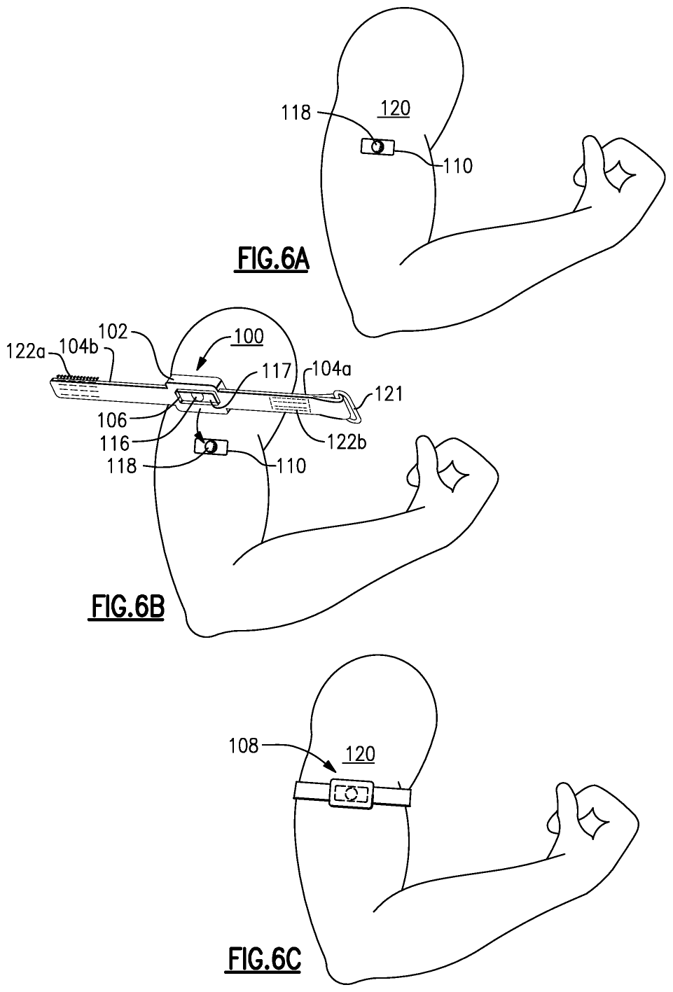

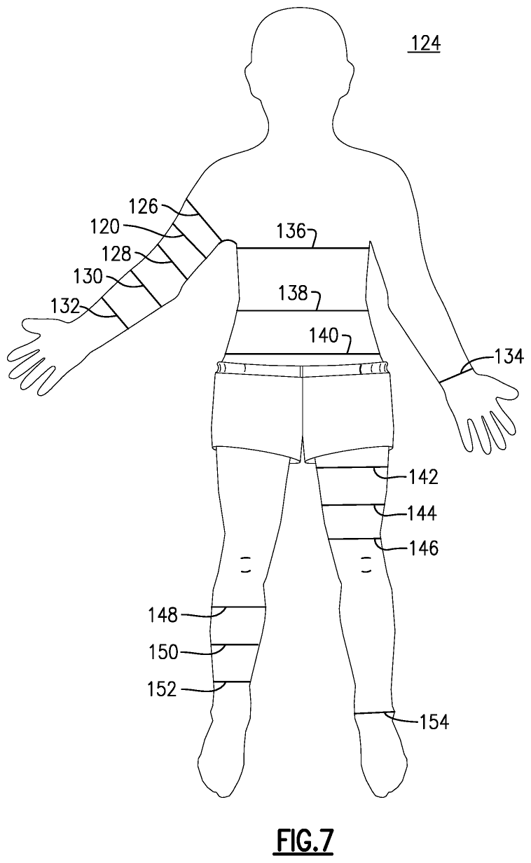

FIG. 6A is a pictorial representation of the patch portion 110 of FIGS. 4 and 5 as placed for use on a middle upper arm location 120 of a user of the present disclosure according to the teachings hereof. As discussed below in conjunction with FIG. 7 hereof, the body location of the two-piece device of the present disclosure may be selected of a number of different body location with such selection being made according to the use application and as such the straps or bands 104 of the bracelet portion may be configured accordingly.

Thus in this use example, as shown in continuing progression in FIG. 6B, the matching bracelet portion 100 is shown in motion as being placed in alignment over the corresponding patch portion 110 where the dome section 118 of the patch portion 110 is shown as aligned for engagement with the dimplet 116 formed in the upper surface 117 of the socket 106 of the central member 102 of the bracelet portion 100. In this embodiment, one of the straps 104a is provided with a clasp, buckle, ring 121 and the other strap 104b is provided with a fastener element 122a shown in FIG. 6B as one element of hook and loop fastening material as is well known in the art of strap fastening materials with the other hook and loop element illustrated therein as 122b.

In other embodiments, not shown, the buckle 121 may include a frame with a bar having a prong attached thereto with the fastener element 122a then being simply a series of progressing holes formed in the strap 104b as is well known in the art of waist belts and watch straps, for example, without the need for the corresponding hook and loop element 122b. Thus in this manner, the patch portion 110 may be seated within the socket 106 with the dome section 118 in register with the dimplet 116 as its corresponding mating element and the straps 104a and 104b then secured together to thereby achieve assembly of the present wearable two-piece integrated bio-patch and bio-bracelet system. And as would be apparent to those skilled in the relevant arts hereof, the coupling between the two principal components hereof can be magnetic, electro-magnetic, electrical, or any combination thereof as well as mechanical.

Thus for example, the patch portion 110 may include a passive RFID tag with a coil and the bracelet portion 100 may include a battery with a circuit for directing current flow such that when the coil and circuit are placed in close physical proximity, with EM coupling thereby provided between the two components, the circuit may induce a corresponding current flow in the coil of the RFID tag so that the RFID tag may thereby broadcast over short range its ID signal. Thus with the above as further provision according to the teachings hereof with same including all other such thereby suggested, FIG. 6C is a pictorial representation of a two-piece system 108 shown in a final coupled condition ready for use as deployed on the middle upper arm location 120 of the user in this particular body-placement example.

Now with reference to FIG. 7, there is shown a body schematic of a prospective user of the present disclosure illustrating several different body locations for deployment of the present disclosure according to the specific embodiment and application for each thereof. Different applications may include sports and fitness such a long distance running, swimming or cycling; clinical diagnostics such as infectious disease detection or non-invasive symptom detection such as fever detection, for example; health and wellness applications such as for use in nursing homes or other long-term care facilities; life styles applications such as human fertility or sober driving; emergency medical such as during ambulance transport of a patient or during long-term ICU care; and such other applications as disclosed in commonly assigned patent applications relating hereto.

Thus in FIG. 7, there is shown the middle upper arm location 120 discussed above in conjunction with FIGS. 6A to 6C and in addition thereto, such other body locations as may be desired according to the application including a high upper arm location 126, a lower upper arm location 128, an upper forearm location 130, a middle forearm location 132, or a location around the wrist as shown at a wrist location 134. In addition to these locations, the present disclosure may be advantageously deployed around a user's torso or upper body such as at a chest location 136, a mid-trunk location 138, or an abdominal location 140 where the patch portion 110 may be placed initially on the front, side, or back of the user. As for leg locations, the present two-piece device may be advantageously deployed around an upper thigh location 142, a mid-thigh location 144, a lower thigh location 146 just above the knee, an upper calf location 148 just below the knee, a mid-calf location 150, and a lower calf location 152. And in certain other applications, the preferred body location may be an ankle location 154 just above the user's ankle bone.

Thus with reference to all the FIGS. 1 to 7 discussed above, the present disclosure is directed to an integrated patch and bracelet system wearable on a user to perform a diagnostic function, the system comprising i) a bracelet portion including a central housing member and a pair of straps extending there from with each strap having an end, the central housing member having a socket formed in a back side thereof and configured to contain a plurality of electronic components, and ii) a patch portion configured to contain at least one diagnostic component and having a peripheral edge that conforms in shape to the socket formed in the central housing member so that when the patch portion is applied to a skin surface of a user and the bracelet portion is positioned over the patch portion with the ends of the straps secured together around a body part of the user, the patch portion is seated in register with the socket in the central housing member thereby allowing interoperability between the at least one diagnostic component in the patch portion and at least one electronic component in the central housing member.

On one preferred embodiment of this system, a dimplet is formed in an upper surface of the socket of the central housing member and advantageously in conjunction therewith, a dome section is formed on a top surface of the patch portion, the dome section containing therein at least a portion of the at least one diagnostic component in the patch portion. Thus in the manner, when the bracelet portion is positioned over the patch portion with the ends of the straps secured together around the body part of the user, the dome section of the patch portion is seated in register with the dimplet formed in the socket in the central housing member thereby allowing interoperability between the portion of the at least one diagnostic component in the dome section and the at least one electronic component in the central housing member. And as described above, the body part of the user may be associated with a body location such as a middle upper arm location, a high upper arm location, a lower upper arm location, an upper forearm location, a middle forearm location, a wrist location, a chest location, a mid-trunk location, an abdominal location, an upper thigh location, a mid-thigh location, a lower thigh location, an upper calf location, a mid-calf location, a lower calf location, or an ankle location.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” The word “coupled”, as generally used herein, refers to two or more elements that may be either directly connected, or connected by way of one or more intermediate elements. Additionally, the words “herein,” “above,” “below,” and words of similar import, when used in this application, shall refer to this application as a whole and not to any particular portions of this application. Where the context permits, words in the above Description using the singular or plural number may also include the plural or singular number respectively. The word “or” in reference to a list of two or more items, that word covers all of the following interpretations of the word: any of the items in the list, all of the items in the list, and any combination of the items in the list.

The above detailed description of embodiments of the invention is not intended to be exhaustive or to limit the invention to the precise form disclosed above. While specific embodiments of, and examples for, the invention are described above for illustrative purposes, various equivalent modifications are possible within the scope of the invention, as those skilled in the relevant art will recognize. For example, while processes or blocks are presented in a given order, alternative embodiments may perform routines having steps, or employ systems having blocks, in a different order, and some processes or blocks may be deleted, moved, added, subdivided, combined, and/or modified. Each of these processes or blocks may be implemented in a variety of different ways. Also, while processes or blocks are at times shown as being performed in series, these processes or blocks may instead be performed in parallel, or may be performed at different times.

The teachings of the invention provided herein can be applied to other systems, not necessarily the system described above. The elements and acts of the various embodiments described above can be combined to provide further embodiments.

While some embodiments of the inventions have been described, these embodiments have been presented by way of example only, and are not intended to limit the scope of the disclosure. Indeed, the novel methods and systems described herein may be embodied in a variety of other forms; furthermore, various omissions, substitutions and changes in the form of the methods and systems described herein may be made without departing from the spirit of the disclosure. The accompanying claims and their equivalents are intended to cover such forms or modifications as would fall within the scope and spirit of the disclosure.

1. An integrated patch and bracelet system wearable on a user, the system comprising:
a bracelet portion including a central housing member and a pair of straps extending therefrom with each strap having an end, the central housing member having a socket formed in a back side thereof and configured to contain a plurality of electronic components; and
a patch portion configured to contain at least one diagnostic component to perform a diagnostic function and having a peripheral edge that conforms in shape to the socket formed in the central housing member so that when the patch portion is applied to a skin surface of a user and the bracelet portion is positioned over the patch portion with the ends of the straps secured together around a body part of the user, the patch portion is seated in register with the socket in the central housing member thereby allowing interoperability between the at least one diagnostic component in the patch portion and at least one electronic component in the central housing member.

2. The system according to claim 1 further including a dimplet formed in an upper surface of the socket of the central housing member.

3. The system according to claim 2 further including a dome section formed on a top surface of the patch portion, the dome section containing therein at least a portion of the at least one diagnostic component in the patch portion.

4. The system according to claim 3 wherein when the bracelet portion is positioned over the patch portion with the ends of the straps secured together around the body part of the user, the dome section of the patch portion being seated in register with the dimplet formed in the socket in the central housing member thereby allowing interoperability between the portion of the at least one diagnostic component in the dome section and the at least one electronic component in the central housing member.

5. The system according to claim 1 wherein the body part of the user is associated with a body location.

6. The system according to claim 5 wherein the body location is an arm location.

7. The system according to claim 6 wherein the body location is a middle upper arm or a high upper arm location.

8. The system according to claim 6 wherein the body location is a lower upper arm location.

9. The system according to claim 6 wherein the body location is an upper forearm location.

10. The system according to claim 6 wherein the body location is a middle forearm location.

11. The system according to claim 6 wherein the body location is a wrist location.

12. The system according to claim 5 wherein the body location is a torso location.

13. (canceled)

14. (canceled)

15. The system according to claim 5 wherein the body location is a leg location.

16. (canceled)

17. (canceled)

18. (canceled)

19. (canceled)

20. (canceled)

21. The system according to claim 15 wherein the body location is an ankle location.

22. A wearable bracelet comprising:
a central housing member having a socket formed in a back side thereof and configured to contain a plurality of electronic components, the central housing member further configured to receive a patch having a diagnostic component for providing a diagnostic function, the socket dimensioned such that a peripheral edge of the patch substantially conforms in shape to the socket so that when the patch is applied to a skin surface of a user and the bracelet is positioned over the patch, the patch is seated in register with the socket in the central housing member thereby allowing interoperability between the diagnostic component in the patch and at least one electronic component in the central housing member; and
one or more straps extending from the central housing member and configured to allow the bracelet to be secured to a body part associated with the skin surface of the user.

23. The bracelet according to claim 22 wherein the central housing member further includes a dimplet formed in an upper surface of the socket.

24. The bracelet according to claim 23 wherein the dimplet of the socket is dimensioned to index with a dome section formed on a top surface of the patch.

25. A patch comprising a diagnostic component for providing a diagnostic function, the patch having a shape that substantially conforms to a socket of a central housing member when the patch is seated in register with the socket, the central housing member having a plurality of electronic components and being a part of a bracelet, so that when the patch is applied to a skin surface of a user and the bracelet is positioned over the patch, the patch being seated in register with the socket in the central housing member allows interoperability between the diagnostic component in the patch and at least one electronic component in the central housing member.

26. The patch according to claim 25 wherein the patch further includes a dome section formed on a top surface of the patch.

27. The patch according to claim 26 wherein the dome section of the patch is dimensioned to index with a dimplet formed in an upper surface of the socket of the central housing member.
